(19) United States
(12) Patent Application Publication
(10) Pub. No.: US 2016/0256101 A1
(43) Pub. Date: Sep. 8, 2016

(54) Device and System Device and System for Imaging Veins

(71) Applicants: Avraham AHARONI, Rehovot (IL); Anatoli RAPOPORT, Rehovot (IL)

(72) Inventors: Abraham Aharoni, Rehovot (IL); Anatoli Rapoport, Rehovot (IL)

(21) Appl. No.: 15/028,663

(22) PCT Filed: Oct. 7, 2014
(86) PCT No.: PCT/IL2014/050883

§ 371 Date: Apr. 11, 2016

(30) Foreign Application Priority Data
Oct. 14, 2013 (IL) 228868

(51) Int. Cl. A61B 5/00; A61M 25/06; A61M 5/34; A61B 90/13; A61M 5/32

(52) U.S. Cl. CPC A61B 5/489; A61B 90/13; A61M 5/3287; A61M 5/34; A61B 5/0086; A61M 25/0693; A61B 5/742; A61B 2090/306

(57) ABSTRACT

A hypodermic needle device (10) includes a hypodermic needle (1) having a tubular bore and at least one optical waveguide (12, 15) extending along said needle so that a distal end of the at least one optical waveguide is in proximity to a tip of the needle. A first coupler (25) secures the optical waveguide within the needle at the proximal end of the needle, and a second coupler (27) removably secures a proximal end of the optical waveguide to a respective illumination source (14) in order that light will emanate from the distal end of the at least one optical waveguide. The second coupler (27) contains a lens for focusing light from the illumination source through the at least one optical waveguide (12, 15), and the second coupler (27) is remote from the first coupler (25).

### FIELD OF THE INVENTION

The invention relates to vein visualization systems and in particular to a vein visualization system for the assistance in the insertion of hypodermic needles and catheters in venipuncture, phlebotomy and intravenous therapy.

### BACKGROUND OF THE INVENTION

Known in the art are vein visualization systems which can, noninvasively, show images of subcutaneous veins. Two main technologies are used for such visualization: ultrasonic imaging and illumination with red or infrared light. Ultrasonic methods are similar to other ultrasonic imaging methods, with probes that generate and detect ultrasound, and imaging electronics which present an image of the back-reflected insonification. These systems show veins clearly and can therefore be used to guide intravenous needles directly into a vein. Typically, while the image quality obtained with ultrasound is good, ultrasound systems do suffer some practical drawbacks, including a poor image close to the skin surface, relatively high cost and large physical size.

The alternative technology takes advantage of the relatively high absorption of red and near infrared (NIR) light in blood. On a forearm, for example, illuminated with red or NIR light, the veins are seen as dark areas over a background of brighter regions of light scattered from surrounding tissue.

While the optical illumination technology offers a smaller and less costly system, it is limited in the depth to which veins can be visualized. The two main limiting factors for the visualization depth are the relatively large reflection and scatter from the surface of the skin, which necessarily reduce the contrast between the absorbing dark vein areas and the scattering back-reflecting tissue areas. Furthermore, the backscattered light from layers of tissue above a deep vein eventually reduces the contrast of imaged veins, limiting the practical imaging depth possible with this method.

Some implementations of optical illumination technology improve the contrast of the optical absorption image by introducing illumination with polarized light and collection of the image through an orthogonal polarization (which may be linear or circular polarization states). As the light reflected from the surface of the skin mostly remains in the same polarization of the incident illumination, and the light scattered from tissue surrounding the vein has statistically random polarization, such imaging through orthogonal polarization states accentuates the scattered light over the light reflected from the surface of the skin, or, in other words, improves the contrast of the optical absorption image. Nevertheless, even with this improvement, the applicability of optical absorption visualization is limited to the detection of veins in depth not exceeding some 6 or 8 mm. This is a serious practical limitation, as for many purposes deeper veins are of interest.

As indicated above, a primary motivation of the present invention is to assist in the insertion of intravenous needles for venipuncture, phlebotomy and intravenous therapy, where fluids are injected into the blood stream or blood samples are extracted. Modern devices for such operations incorporate a rigid hypodermic needle or intravenous (IV) needle and a flexible catheter placed over the needle. The needle serves to mechanically pierce the skin and tissue and penetrate a vein. Once the assembly is located in the vein, the needle is withdrawn and the flexible catheter is secured inside the vein, serving as a duct for transfer of fluid into the blood stream, or removing blood for testing.

Considering the procedure for insertion of the needle, it is generally divided into two parts. First the person performing the procedure, for example a phlebotomist, searches for a suitable vein. A vein with active blood flow (termed a patent vein), with a relatively large size, and a straight stretch with no bifurcations is sought. In searching for a suitable vein the phlebotomist combines her/his prior knowledge of anatomy and the location of suitable veins, her/his visual image of the patient's veins, and often the tactile feedback of a vein to manual pressure applied to it. Obviously the latter two inputs can often be very limited, especially in elderly or obese patients, those with dark skin or patients who have low blood pressure due to dehydration or other medical conditions. The present invention aims to assist a phlebotomist in the vein-locating operation by providing an enhanced image of potential veins, showing their approximate size, their general layout and the presence of vein bifurcations. Optionally the present invention can also provide direct information on the blood flow in the vein. To a greater extent, the invention aims to assist a phlebotomist in the second stage—the physical insertion of a needle into the selected vein.

In the second part of a conventional procedure, the phlebotomist inserts a needle into the selected vein. This process involves careful aiming of the needle towards the selected vein and navigating its tip towards the center of the vein's width to overcome situations where the vein flexes away at the contact of the needle (a situation called a rolling vein). Once in contact with the vein, the phlebotomist needs to penetrate the frontal vein wall carefully and avoid reaching the opposite vein wall.

U.S. Pat. No. 5,030,207 discloses a device for indicating when an intravenous needle has entered the vein through the use of a solid fiber optic mounted in the needle for showing visual instantaneous vein entry. The distal end of the fiber optic is polished to be flush with the distal point of the needle. The fiber optic is sized to have an outer diameter which fills the internal bore of the needle. On contact with the blood in the vein, the polished distal end of the solid fiber collects ambient light filtered by the blood and transmits it through the solid fiber to a magnifying arrangement located at the rear or proximal end of the fiber optic. The user observes immediate vein entry without any blood flow or exposure to blood. Other embodiments utilize the solid fiber optic itself for piercing the tissue, thus eliminating the needle altogether. It is also possible to rely on ambient light that is collected by the magnifying arrangement and directed into the solid fiber to illuminate the tip, or use supplementary illuminators to increase the illumination. The operator is constrained to view the vein through a narrow field of view and is required to distinguish between relatively small variations in the color of the low light level collected by the small solid fiber's tip and transmitted through the optics of the device.

The system disclosed in U.S. Pat. No. 5,030,207 also appears to require the use of a solid optical fiber, there being no suggestion to use a disposable optical fiber or to illuminate the internal opening of the needle directly without using an optical fiber. The illumination is primarily based on ambient light, although an option for supplement illumination at the distal end of the needle is suggested. The illumination is intended to scatter off the blood in the vein, a portion of which enters the solid optical fiber and appears as a red indication to a user viewing the magnification arrangement. Furthermore, there is no indication of the possibility of using an extended optical fiber to allow a mechanically separate illumination source at a convenient distance. U.S. Pat. No. 4,311,138 likewise discloses a hypodermic needle adapted to emit light from its distal end to facilitate venopuncture under subdued lighting conditions. The needle is used in conjunction with a portable light source, such as a battery handle and lamp, and includes an optical fiber bundle that transmits light from the lamp to the distal end of the needle. A flexible catheter is releasably mounted on the needle and is adapted to be inserted in the vein after the needle has punctured same and thereafter the needle can be withdrawn from the catheter.

The system disclosed in U.S. Pat. No. 4,311,138 appears to require the use of an optical fiber bundle, there being no suggestion to use a disposable single strand optical fiber or to illuminate the internal opening of the needle directly without using an optical fiber. The illumination source considered is a white light lamp with a limited percentage of the light output being coupled into the fiber. The illumination itself is broad band white light which does not serve to accentuate the location of red-absorbing blood vessels. In addition there are no measures included in the disclosure to ensure that fragments of the optical fiber bundle do not break off and remain within a patient's body.

### SUMMARY

It is an object of the present invention to closely assist the delicate needle insertion procedure by providing an optical image of the location of the needle tip to the target vein at all times, and indications on the instance of penetration into the front wall of the vein, where further insertion of the needle should be arrested to avoid damage to the opposite vein wall.

It is a further object of the present invention to monitor the blood flow in a vein in the process of selecting a suitable vein for insertion of the needle.

It is a further object of the present invention to offer similar advantages for automated intravenous needle insertion devices.

In accordance with one aspect of the invention there is provided a hypodermic needle device, comprising:

a hypodermic needle having a tubular bore,

at least one optical waveguide extending along said needle so that a distal end of the at least one optical waveguide is in proximity to a tip of the needle,

a first coupler for securing the at least one optical waveguide within the needle at the proximal end of the needle, and

a second coupler for removably securing a proximal end of the at least one optical waveguide to a respective illumination source in order that light will emanate from the distal end of the at least one optical waveguide; wherein:

the second coupler contains a lens for focusing light from the illumination source through the at least one optical waveguide, and

the second coupler is remote from the first coupler.

Other aspects of the invention are defined by the respective independent claims.

The present invention is designed to extend the visualization ability of red or NIR illumination to detect veins at larger depth and assist in guiding a needle tip to a selected vein. This is accomplished by introducing the illumination source to the tip of the intravenous needle, such that the illumination originates inside the tissue. In this manner interference from the relatively strong reflection and back-scattering from the surface of the skin is completely alleviated. Any such reflection from the skin is directed away from the viewer. Furthermore, the illumination is required to traverse the tissue surrounding the viewed vein only once and not twice (into the tissue and then out again) as is the case with conventional devices, so that for a given illumination level the imaging can be effected at twice the depth. As described in more detail in the following, the proposed device is applicable for use in the vein-search phase and to a greater benefit in the needle insertion phase, assisting both manual and automated IV needle insertion operations.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A to 1D show schematically the main components of a prior art needle/catheter assembly and their implementation in the different steps of insertion of the IV needle;

FIGS. 2A to 2D show schematically the main components of the proposed needle/catheter/optical fiber assembly and their implementation in the different steps of insertion of the IV needle;

FIGS. 3A to 3L show schematically different options for implementation of the proposed needle/catheter/optical fiber assembly;

FIGS. 4A to 4G show schematically different operational modes of the proposed needle/catheter/optical fiber assembly with various accessories that offer increased levels of machine assistance to the manual insertion of the IV needle;

FIG. 5 is a schematic block diagram for a fully automated vein search and needle insertion system; and

FIGS. 6A and 6B show schematically flow charts for the vein search phase and the IV needle insertion phase, respectively.

### DETAILED DESCRIPTION OF EMBODIMENTS

In the following description of some embodiments, identical components that appear in more than one figure or that share similar functionality will be referenced by identical reference symbols.

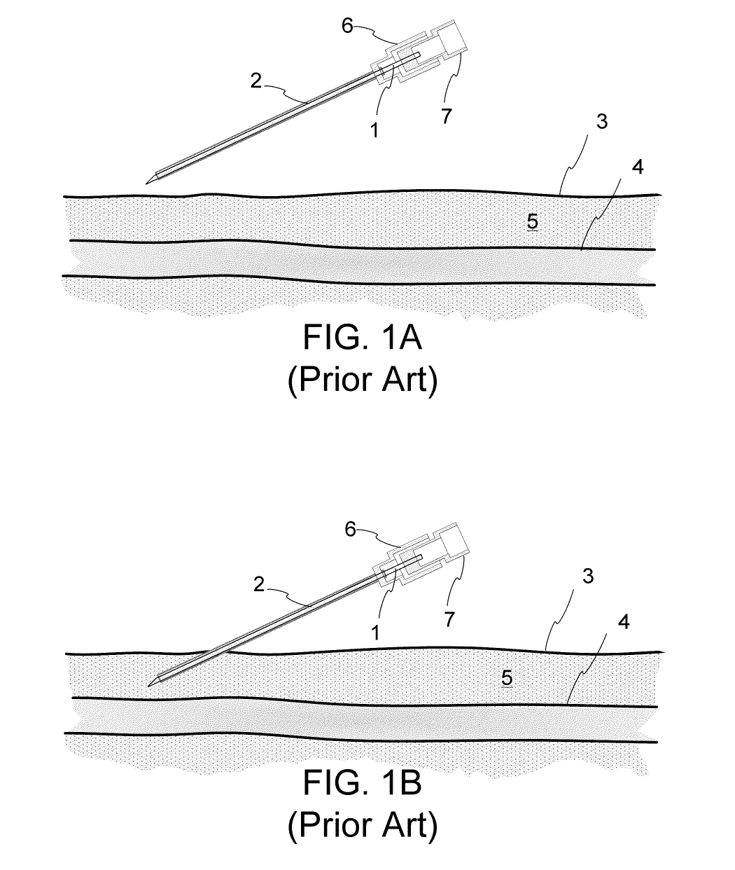

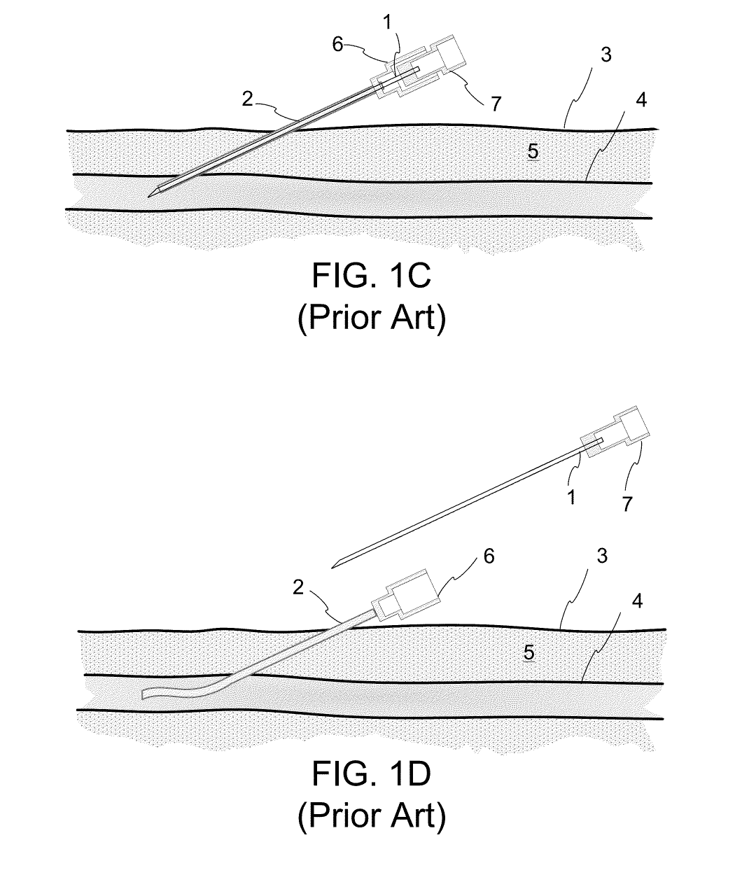

Before describing the invention in detail we consider the state of the art of IV needles and their application shown in FIGS. 1A to 1D. The IV needle assembly comprises a hypodermic penetrating needle 1, and a flexible catheter 2. The assembly is inserted through the skin 3 and into a vein 4 via intervening tissue 5 (FIG. 1B). An important feature of the state of the art needles is the backflow of blood that is visible in a suitable chamber at the proximal end of the needle once the vein is penetrated. This is a clear indication to the person inserting the needle that it has entered the vein, that further penetrating motion must stop and that care has to be taken so as not to puncture the opposite vein wall. Another important feature is the mechanical interface between the respective interlocking mechanical hubs 6 and 7 of the catheter 2 and needle 1, and which also facilitates attachment to a syringe barrel or other tubing by means of a press-fit or twist-on fitting. Once the catheter 2 is inside the vein 4 (FIG. 1C), the needle can be withdrawn as shown in FIG. 1D. The catheter 2, which is now located inside the vein 4, is used as a duct for either removing blood samples for laboratory testing, or for insertion of plasma or other fluids into the vein.

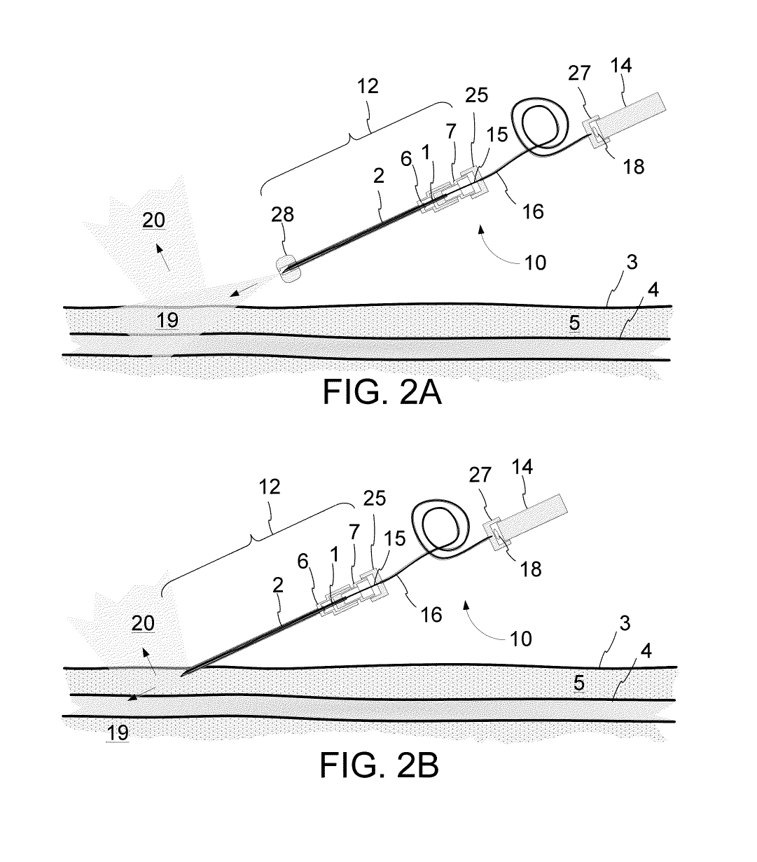

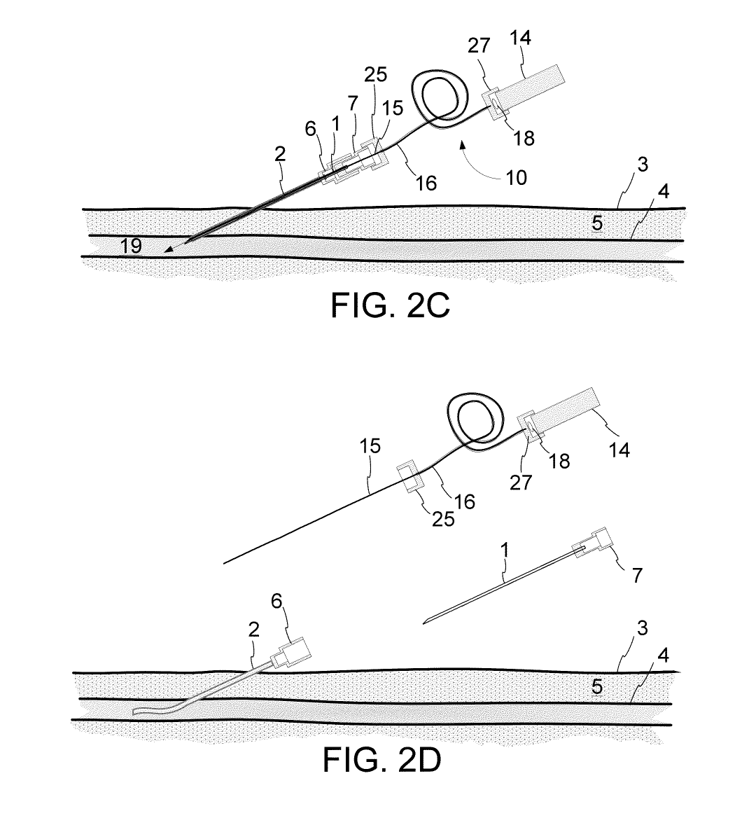

FIGS. 2A to 2D show schematically a hypodermic needle device 10 having a needle/catheter/optical waveguide assembly 12 that is remotely coupled to an illumination source 14 and its mode of implementation according to an embodiment of the present invention. For simplicity we refer to the optical waveguide as a single strand optical fiber, although, as will become apparent in the following, other options are considered. An optical fiber 15 that may be enclosed within a protective sleeve 16 is inserted into a state of the art IV needle 1 and flexible catheter 2 assembly such as shown in FIGS. 1A to 1D of the drawings. The needle 1, catheter 2 and the optical fiber 15 together constitute the needle/catheter/optical fiber assembly 12. The optical fiber is a single strand fiber as opposed to an optical fiber bundle as disclosed in U.S. Pat. No. 4,311,138. The most commonly used needles for drawing blood for blood tests are 21-gauge needles with inner diameter of 0.514 mm, while the most commonly used needles for blood donation are 16- or 17-gauge needles with internal diameters of 1.194 and 1.069 mm, respectively. Such dimensions are much larger than the typical size of a standard optical fiber, in the order of 0.1 mm, allowing the placement of a fiber inside the needle while leaving sufficient room for blood flow within the internal bore of the needle. The illumination source 14 is directed via optics 18 through the optical fiber 15, whose distal tip (i.e. that which is most remote from its illumination source) is close to the tip of the hypodermic needle so that red or NIR light directed through the fiber exits its distal tip to illuminate the vicinity of the hypodermic needle tip. Light exits the optical fiber as a cone of illumination 19, which is reflected and backscattered to form an optical absorption image 20 of veins in its proximity.

The fiber 15 is secured onto the hub 7 of the IV needle 1 by a suitable distal fitting 25 (constituting a first coupler) using a friction mount or screw mount or other mechanical attachment. The proximal tip of fiber 15 is mounted inside a removable fitting 27 (constituting a second coupler), which, in turn is connected to the illumination source 14. The illumination source 14 can be any suitable source such as a solid state laser, a fiber laser, a semiconductor laser, an LED, or other similar light sources. The optics 18 is configured for efficient coupling of the light from the illumination source 14 into the fiber's proximal end.

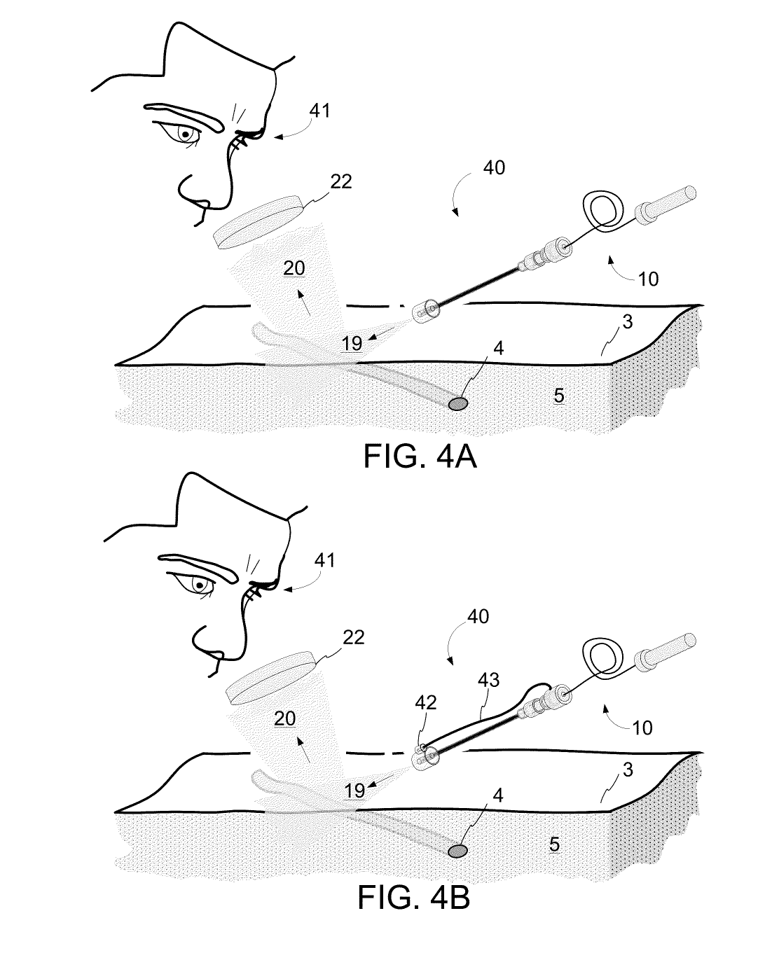

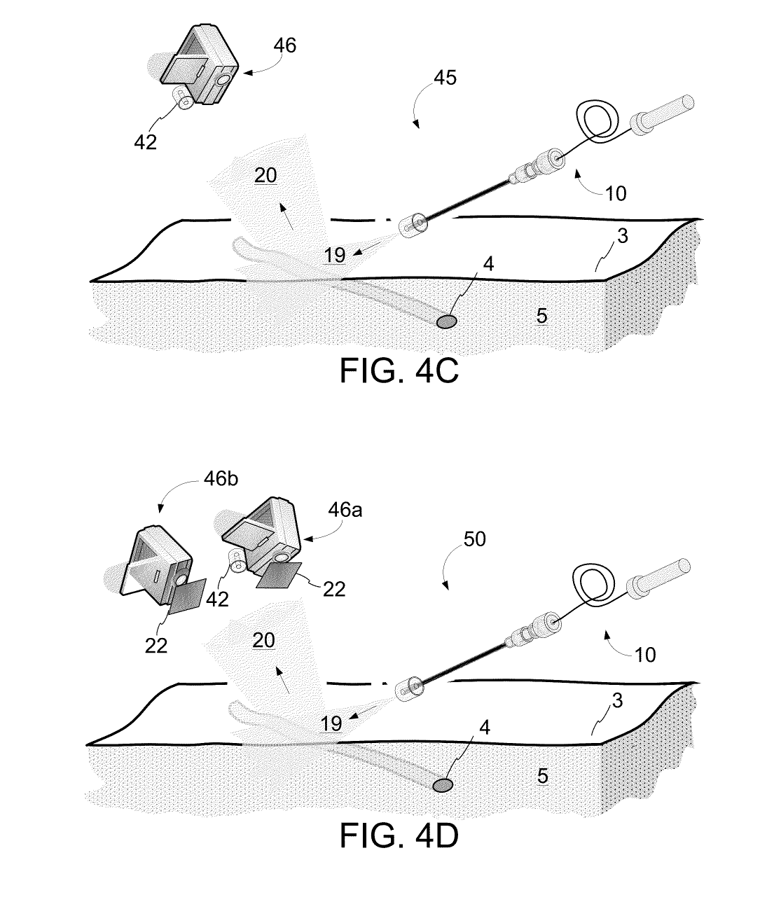

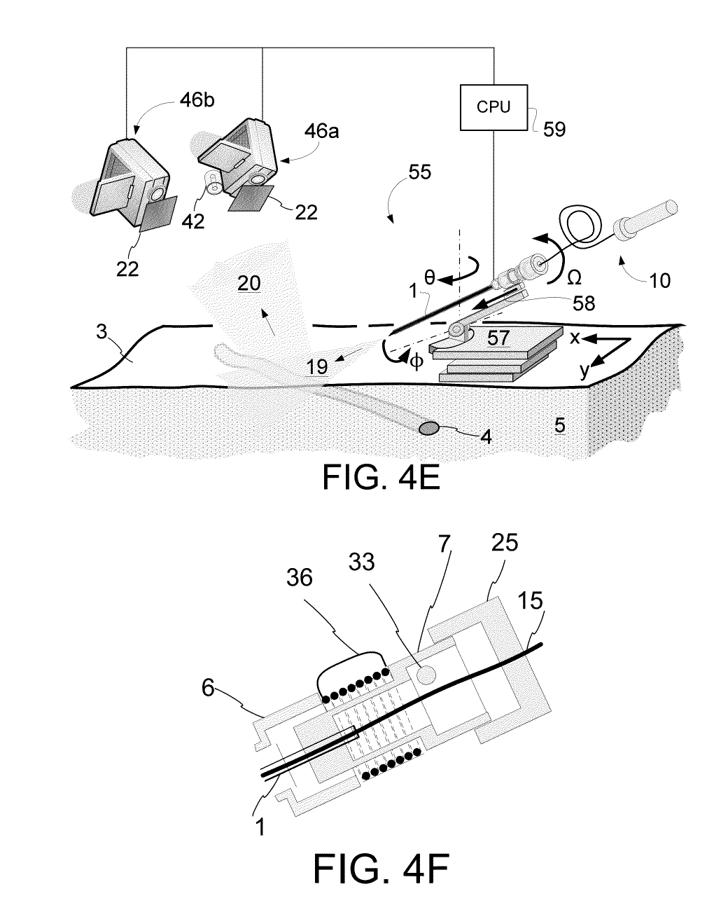

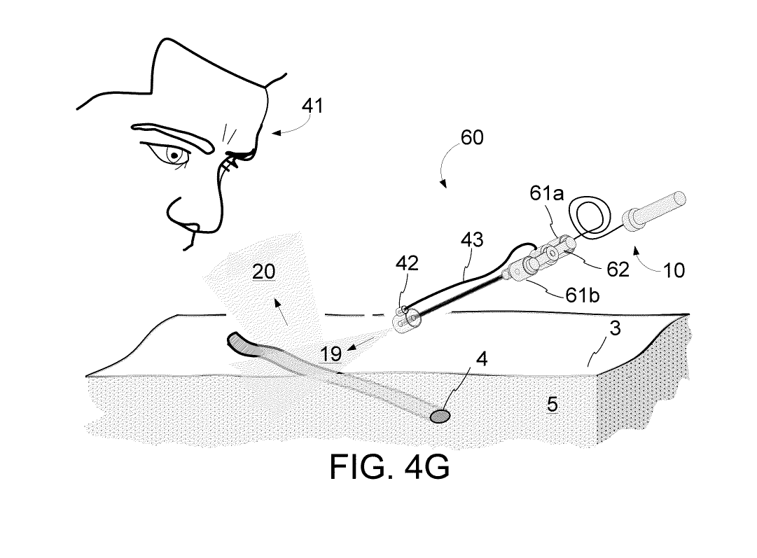

As seen in FIGS. 2A to 2D, the optical fiber 15 is inserted into the tubular bore of the needle 1 with its distal tip located close to the tip of the needle. The entire assembly is now inserted through the skin 3 and tissue 5 (FIG. 2B) into a vein 4 (FIG. 2C). The fiber is fed light from its proximal end from illumination source 14, and illuminates the vicinity of the needle's tip during the entire process. The vein can be viewed as a dark absorbing region on a brighter background of light backscattered off tissue. In the search phase, the proposed arrangement can be used to assist in searching for a suitable vein. Here a needle tip protector 28 can optionally be added to the assembly to avoid accidental pricking of the skin with the needle tip (FIG. 2A). In this phase, vein imaging is similar to that of known state of the art devices: red or NIR light is absorbed by the blood in the veins, which are displayed as dark patterns on the brighter background of light scattered from the surrounding tissue. As best seen in FIGS. 4A to 4G, the image is viewed in the reflected and back-scattered light 20, either with the bare eye for visible red illumination, or with a suitable camera for red or NIR illumination (neither eye nor camera are shown in FIGS. 2A to 2D). The arrangement may optionally include orthogonal-polarization viewing, where the illumination is polarized in one state and an orthogonal polarizer 22 (shown in FIGS. 4A and 4B) is placed in front of the viewer or viewing camera. For this purpose an inherently polarized source may be used, or a polarizer may be inserted in the source assembly. Alternatively a fiber polarization controller can be inserted into the fiber itself anywhere along its free length between the first coupler 25 and the second coupler 27.

Once a vein is selected, any optional needle tip protector 28 is removed, and the insertion phase ensues wherein the assembly is used to pierce the skin and navigate the needle towards the selected vein (FIG. 2B). The illumination 19 from the distal fiber tip now emanates from within the medium of tissue, producing no reflection from the skin surface, and reducing the distance the light has to travel to be viewed. Consequently deeper veins can be visualized with the same arrangement as in the pre-insertion phase shown in FIG. 2A. Moreover, the light emanating point clearly marks the position of the tip of the needle, so that the position of the tip relative to the vein can be visualized. As the light emanating point travels closer to the target vein, the contrast of the vein's dark pattern over the background of light backscattered from the tissue increases. In addition, the light emanating point at the needle's tip and the vein can be viewed at an off-normal perspective providing clear indication to the separation between the two. This visualization facilitates the accurate guidance of the needle tip towards the selected vein, while monitoring their relative transverse location (by viewing at a right angle to the skin surface) as well as their relative separation (by viewing at an off-normal angle to the skin surface). Furthermore, as the light emanating point draws near to the target vein the traverse dimension of the illuminating halo decreases monotonically as a larger portion of illumination cone 19 is blocked directly by the absorbing blood in the vein. Indeed, when the tip of the needle is just at the front wall of the vein, a large portion of the illumination 19 is absorbed by the blood inside the vein, with essentially all the illumination being blocked once the needle's tip pierces the front wall of the vein and the point from which the illumination cone 19 emanates is immersed in blood. This situation, schematically indicated in FIG. 2C, produces a very short illumination cone 19 and essentially no reflected/backscattered light 20.

The above description illustrates how the present invention facilitates accurate guidance to a selected target vein, both in the lateral aspect as well as in depth, and provides a distinct indication of the penetration of the front wall of the vein. The latter feature is of primary importance in alerting the person (or robot) inserting the needle to stop moving the needle inward to avoid damage to the back wall of the vein. This feature is available in the present invention in addition to the blood backflow on penetration of the vein as occurs in state of the art devices since the arrangement of the fiber inside the needle leaves sufficient room for blood to backflow.

Once the vein is penetrated and the catheter is in place, the optical fiber 15 and its assembly can be removed either by itself or together with the needle (FIG. 2D). To remove the fiber 15 itself, the fiber distal fitting 25 is released from the needle hub 6, and the fiber pulled away and out of the needle. To remove the fiber and needle together, which, as explained in the following can both be discarded, the needle is released from the catheter hub 7 as is common to various devices in use. At this point the flexible catheter 2 remains inserted in the vein, as with prior art devices (FIG. 2D), and suitable tubing or other devices, as known in common practice, can be used to remove blood samples or inject fluids. In this respect the present invention does not modify any of the commonly accepted procedures of venipuncture, phlebotomy or intravenous therapy but rather provides for improvements in the location of a target vein in the vein search phase, and facilitates the navigation and insertion of the needle in the insertion phase of the procedure.

It is a primary objective of the present invention to provide for a physically small device that can be handled by a phlebotomist, for example, with essentially no added difficulty. Therefore the proposed device is designed as a small addition to state of the art needle/catheter assemblies. In its basic form the needle/catheter assembly is modified only with an additional distal fitting 25. The fiber 15 and its protective sleeve 16 are small and flexible and essentially do not introduce additional handling difficulty to a phlebotomist. The fiber 15 can be made sufficiently long to allow the illumination light source 14 to rest at a comfortable distance, in the lap of the phlebotomist, on a nearby table, on the patient's bed or chair or even on the body of the patient. The source assembly itself can be battery operated, and be no larger than a small laser pointer. As described below, some implementations of the present invention do require modifications to the needle/catheter assembly itself. Such changes are not considered to detract from the benefit of the invention since the entire needle/catheter/fiber assembly 12 may be in the form of a unitary, disposable, sealed, sterilized package, to be opened, ready for use, immediately prior to the insertion of the needle into the vein.

The fiber, and especially the fiber distal region, which is exposed to body fluids of a patient, requires, as a minimum, sterilization. Preferably, the fiber and its supporting parts, referred to as the fiber assembly, can be made disposable, replacing the fiber tip with every IV insertion; in this case the fiber proximal fitting 27 to the illumination source is removed and the source assembly can be reused. Providing for a discardable fiber assembly offers two practical advantages in addition to the alleviation of the need to sterilize it after every use: as noted above, it can readily be supplied assembled with the needle/catheter assembly in one sterile package to be opened just before use. It is also discardable together with the needle, as described above, so the fiber/needle assembly may be simplified: the fiber distal fitting 25 may be molded together with the needle hub 7.

In any case, in one embodiment the present invention provides a personal, pocket-size device that will become a personal accessory for medical staff, much like the stethoscope. The personal vein visualizer can serve the phlebotomist in drawing blood tests, nurses and physicians in inserting intravenous catheters in a hospital ward or in the emergency room, as well as paramedics treating injuries in the field. In addition to its small physical size the device is also designed to be low cost, comprising a small number of low cost components: a semiconductor laser source, a short optical fiber and plastic molded casings and tubings. Variations of the personal vein visualizer can be devised as sensors for increasing the automation level of fixtures or automated machinery for replacing various manual operations of the procedure of inserting a needle into a vein.

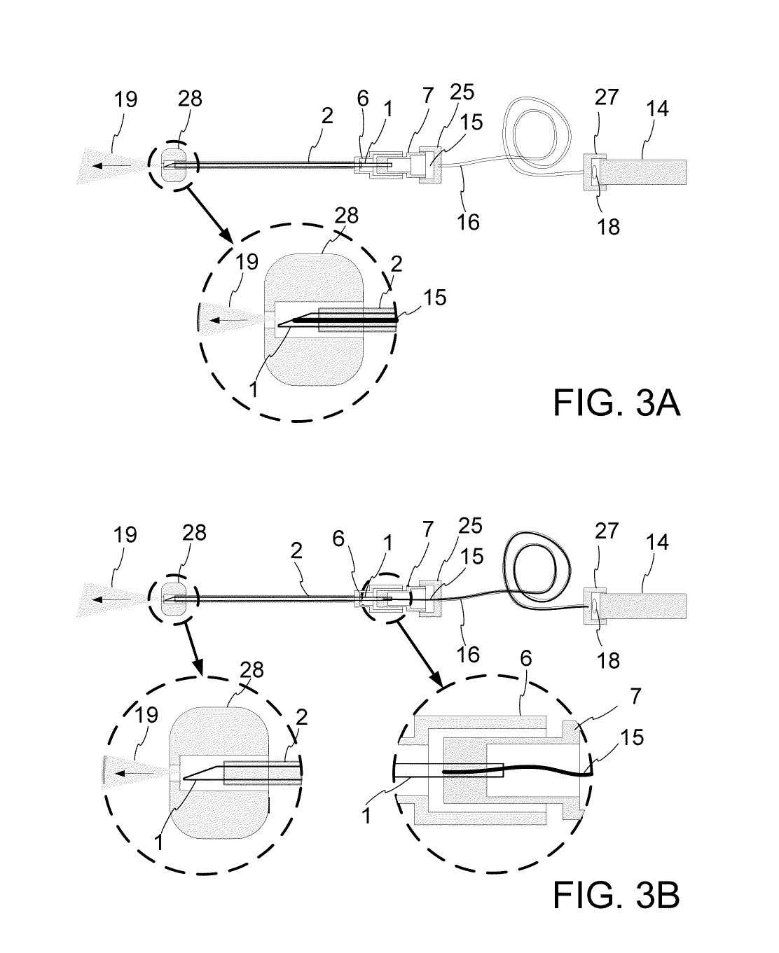

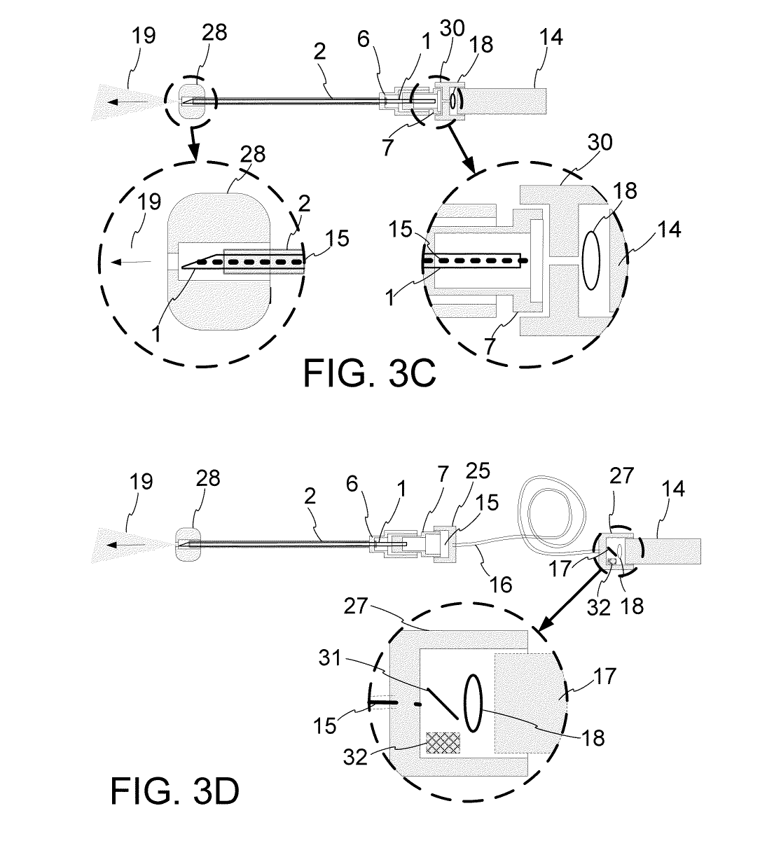

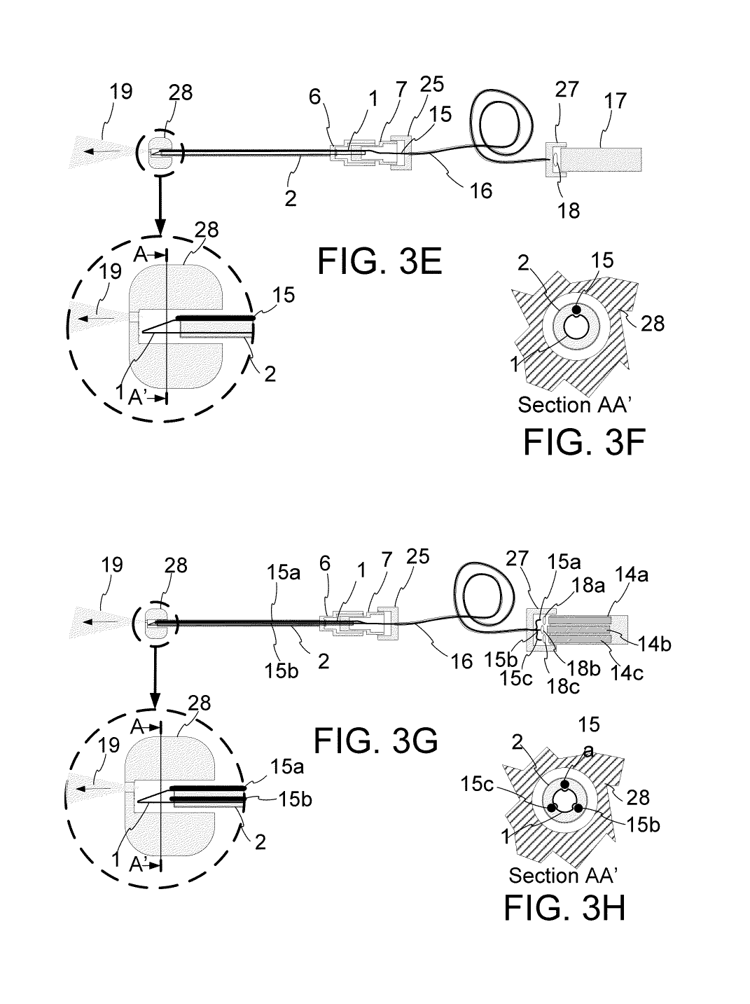

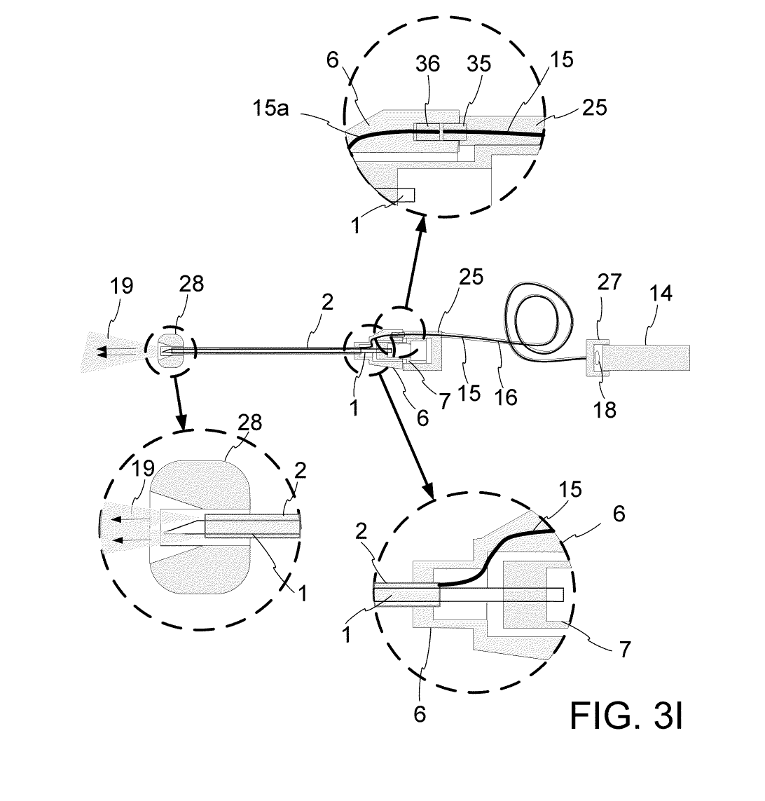

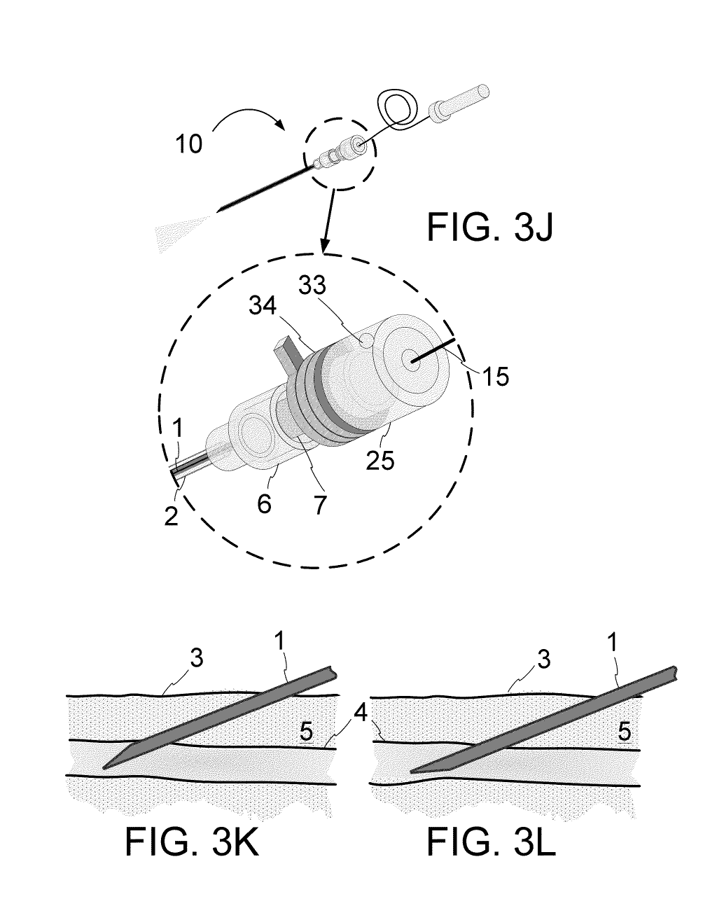

We now consider several modifications to the basic personal vein visualizer described above as depicted schematically in FIGS. 3A to 3I. Note that the optional polarization controller, which can be inserted into the fiber itself anywhere along its free length between the first coupler 25 and the second coupler 27, is not shown in these figures.

FIG. 3A shows schematically the basic personal vein visualizer described above. The optical fiber 15 is inserted into the needle 1 until its distal end reaches close to the tip of the needle as shown in the enlarged view of the tip in FIG. 3A. An alternative arrangement is shown in FIG. 3B where the distal tip of the fiber 15 ends close to the proximal end of the needle 1. This is depicted in the enlarged views in FIG. 3B showing the distal tip of the needle with no internal fiber and the fiber ending close to the proximal end of the needle. In this arrangement the internal surfaces of the needle serve to guide the illumination toward the distal tip of the needle where the illumination 19 exits the needle in a similar form to that in the basic option of FIG. 3A. A third alternative implementation is illustrated schematically in FIG. 3C. Here the illumination source 14 is mounted directly onto the needle hub 7 with the aid of a mounting adapter 30. The optical fiber 15 is shown as a dotted line since it may or may not be included. Thus, in one embodiment, it is omitted and light is shone directly into the internal opening of the needle to be guided along the needle through reflections off its internal surfaces. An optical focusing element 18, depicted in FIG. 3C as a lens, serves to focus the light into the needle's proximal opening. Alternatively an optical fiber can be included to guide the light from the source to the needle, either ending near to the needle's proximal end as in FIG. 3B, or at its distal end as in FIG. 3A.

A fourth alternative, additional to the three configurations described in FIG. 3A through 3C, is described schematically in FIG. 3D. Here a beam splitter 31 is incorporated into the illumination source assembly to redirect a portion of any back-reflected light in the optical fiber 15 to a detector 32. Such back reflection traveling in the optical fiber can be used as an alternative or additional indication for penetration into the front wall of the vein. As long as the needle's tip is in the tissue surrounding a vein, a portion of the backscattered light will enter the fiber and travel backwards towards the beam splitter 31 and the detector 32. On penetration into the vein, the blood absorbs the illumination, significantly reducing backscatter back into the fiber. The blood also wets the distal end of the fiber reducing the internal reflection from this interface. Both of these effects introduce a significant drop in the backscattered light travelling backwards in the fiber. A similar arrangement with a detector that monitors back reflected light is also possible with the needle-wall guiding implementation of FIG. 3C. Optionally and additionally an optical detector may be included to monitor the presence of blood in the backflow chamber of the needle. Optionally a light source may also be included to illuminate the backflow chamber for an improved optical signal. Such a sensor can provide an additional or alternative alert for entry of the needle tip into the vein.

In a fifth alternative, the schematic arrangement of FIG. 3A is modified in that the optical fiber inserted into the needle and extending along a substantial length of the needle, or indeed its entire length, is cemented on to the side of the needle wall. This may be implemented by introducing the optical fiber into the needle together with an insert spacer, so that the fiber can be cemented to one side of the needle duct, leaving part of it open for blood backflow. Alternatively, the fiber and cement can be forced to one side of the internal duct of the needle by placing the assembly with uncured cement into a centrifuge. The cement is cured when the centripetal forces force the cement and fiber to one side of the needle duct. The advantage of cementing the fiber to the side of the internal duct of the needle relates to the danger of the fiber breaking and a portion of it remaining inside the body of a patient. One consequence of this implementation is that cementing the fiber to the needle does not allow the separation of the needle/fiber assemblies as anticipated in FIG. 2D. In this implementation the needle and fiber assemblies are inseparable, so that the two need to be removed together from the catheter/needle/fiber assembly 12 after positioning the catheter inside the vein.

A sixth alternative is shown schematically in FIG. 3E. Here the optical fiber is attached to the needle from its outside as depicted in FIG. 3F showing a section along AA′ of the enlarged image of the tip of the needle fiber assembly in FIG. 3E. The fiber may readily be cemented to the needle to alleviate the danger of loss of a fiber fragment in a patient's body. The fiber may be cemented on to a standard needle, or optionally and alternatively cemented onto a suitable recess or groove introduced along the length of the needle as depicted in Section AA′ of the enlarged image of the tip of the needle fiber assembly in FIG. 3E. Such a groove can be formed with a suitable press mould. Additionally and alternatively, there may be attached to the assembly more than one fiber, each being a separate single optical fiber strand. FIG. 3G shows a three-fiber arrangement, of which an enlarged section along line AA′ is depicted in FIG. 3H showing a detail of the tip of the needle fiber assembly. The fibers may be distributed evenly around the circumference of the needle in suitable grooves (15a through 15c in FIG. 3H), or mounted in the same groove, or cemented on to the unmodified needle surface. The multiple fibers, fed by independent illumination sources 14a through 14c with suitable focusing optics 18a through 18c, can be used to increase the illumination power relative to that of a single fiber. Alternatively different fibers can be used to illuminate at different wavelengths so as to allow, for example, a combination of red and infrared illumination. Such combined illumination permits combined viewing with an eye and a camera and improves the performance of a system having only one of these viewing options.

A seventh alternative is shown schematically in FIG. 3I. Here the catheter tubing 2 serves as the waveguide for transmitting light to the tip of the hypodermic needle. Light coupled into the proximal end of the catheter tubing 2 emanates at its distal end close to the tip of the needle, forming essentially an illumination ring 19. Although physically more expanded, such illumination is sufficient to perform all the function described above in relation to the more confined illumination generated by a single fiber. The light is coupled into the catheter tubing 2 by an optical fiber 15 that is adapted to inject light into the wall of the catheter tubing at its proximal end. Such a fiber may be embedded in the wall of the catheter 2, extending a significant length along the tubing. Indeed, the fiber may optionally extend the full length of the catheter tubing, effectively offering a similar point source illumination as in the implementation of FIGS. 3G and 3H. Alternatively the fiber may extend a distance along the catheter wall coupling light into it; this light is guided along the catheter tubing 2 to emanate as essentially the above mentioned annular illumination. Alternatively more than one fiber may couple light into the catheter wall (not illustrated in FIG. 3I). As described above in connection with FIGS. 3G and 3H, such multiple fibers, fed by independent illumination sources with suitable focusing optics, can be used to increase the illumination power of a single fiber, or, alternatively, illuminate at different wavelengths so as to allow, for example, a combination of red and infrared illumination.

One challenge of the configuration of FIG. 3I relates to the mechanical coupling of the source fiber or fibers into the catheter hub 6. One possibility (not shown in the figures) is to embed the fibers into the catheter hub 6 and extend the fibers continuously to the second coupler 27. In this option it is not possible to remove the fiber assembly from the catheter after the catheter is positioned in the vein. This is inconvenient when the catheter is required for extended operation as, after the catheter is located in the vein, the fiber is a mechanical disturbance. One possibility to overcome this limitation is to break the fiber off the catheter hub 6. A more elegant solution is to provide two separate fibers: one fiber 15, extending from the second coupler 27 to the first coupler 25 and the other fiber, 15a, extending from the proximal face of the catheter hub 6 to the catheter tubing 2. Mechanical centering and alignment elements, similar to those used in standard optical fiber connectors, are provided: an element 35 mounted onto the first coupler 25 to center and align the distal end of the fiber connected to the illumination source 15; and an element 36 mounted onto the catheter hub 6 to center and align the proximal end of the fiber connected to catheter tubing 15a; The mechanical centering and alignment elements 35 and 36 ensure, on the one hand that when assembled the two fibers 15 and 15a are aligned and illumination is transferred efficiently from one fiber to the other, and, on the other hand, can be separated once the catheter is positioned in the vein. The mechanical elements can be held together with press-fit or twist-on fitting or with the aid of a breakable pin or latch so that it is possible to manually separate the first coupler 25 from the catheter hub 6.

Two additional optional features to the devices described above are shown in FIGS. 3J through 3L. The first is a blood backflow detector 33 which, as described above, serves to identify the backflow of blood into the blood backflow chamber. This is an indication that the vein has been penetrated. In addition there may optionally be provided a spring-loaded mechanism 34 to rotate the needle 180° about its axis (shown as Ω in FIG. 4E). This rotation inverses the needle tip, which is positioned with its sharpest portion first for ease of the initial penetration the vein, as shown in FIG. 3K, to the orientation of FIG. 3L with the sharpest tip of the needle upward. The latter orientation is not optimal for additional penetration of the needle and in that position the needle is less likely to pierce the back wall of the vein. The spring-loaded mechanism 34 allows an operator to rotate the needle automatically by releasing a latch that secures the needle in one orientation. On release of the latch, the needle rotates by approximately 180° to the “pierce-safe” orientation. In automated applications, such as robotic control of the needle (described below), this operation may be performed on command of a central processor on receipt of a positive signal from the blood backflow detector 33 indicating penetration of the vein. This spring loaded axis is applicable to the full range of systems spanning from fully manual operation through increased automation levels to the fully automated robotic system, all described below.

We now consider several vein visualization systems, described schematically in FIGS. 4A through 4G, utilizing the needle/catheter/optical fiber assembly described above.

A basic system 40 is shown schematically in FIG. 4A, where the needle/catheter/optical fiber assembly 10 is deployed manually and the veins viewed with the unaided eyes of an operator 41. For this purpose red illumination is used. The illumination source can be battery operated. Optionally, the illumination source may be polarized and the operator 41 wears eye glasses, or a similar head-worn device, with an orthogonal polarization. In this case, the system may optionally include a polarization controller so that the operator can optimize the contrast of the vein images (not shown). Alternatively the polarization of the polarization device may be manually adjustable. FIG. 4A shows the system 40 in the first phase of the operation—searching for a suitable vein. The additional steps of the operation, shown in FIG. 2B through 2D are performed in a manner similar to the description referring to these figures. It is noted that the system inherently offers the operator a perspective view of the relevant vein. Using both eyes, the operator can discern both lateral offset between the needle tip and the vein as well as depth separation between the two. This is similar to the 3D image offered by use of two cameras as depicted in FIG. 4D and further detailed below.

An alternative system is described schematically in FIG. 4B. Here a Doppler detector 42 is added to the needle tip protector (28 in FIG. 2A). In one form, this detector collects light reflected from the blood in the selected vein. Although the reflected light from the blood itself is very weak, the inventors have found that under certain conditions, it is possible to detect a Doppler shift in the reflected light which is indicative of the flow rate of the blood in the selected vein. Such detection is indicative of the potency of the selected vein. Here the detector 42 is coupled via a cable 43 through the fiber distal fitting 25 (FIG. 2A) through the fiber protective tubing 16 (FIG. 2A) back to the housing of the illumination source 14 (FIG. 2A). In this configuration the optical Doppler shift detector, comprising essentially no more than a photodetector, can be discarded with the disposable fiber assembly. Alternatively the optical Doppler detector can be connected directly via the cable 43 to the housing of the illumination source 14 and be used for repeated operations. Alternatively the reflected light is detected with the internal detector 32 in FIG. 3D.

The light detected with the optical Doppler detector is amplified, filtered and the resulting signal processed with suitable electronics. The output of the electronic processing is displayed to the operator, either in the form of text or an analog intensity indications such as bar display or other visual display means indicating the detected blood flow rate.

In an alternative form of implementation the Doppler detector 42 is an ultrasonic transceiver which transmits an ultrasonic signal in the direction of the vein and detects the reflected ultrasonic signal which is Doppler shifted in correspondence to the blood flow rate in the vein. In this case the ultrasonic transceiver should be placed in contact with the patient's skin, and optionally coupling fluid applied to couple the ultrasound into and out of the body of the patient. Such an ultrasonic Doppler detector can be used for repeated operations and be either wired directly to the illumination source enclosure (for power and display) or provided in a separate enclosure with independent power and display. In either case, the ultrasonic Doppler detector can be provided with a disposable plastic or nylon cover to alleviate the need for sterilizing it after each use. There is a distinct tradeoff between the use of an optical Doppler detector, which is smaller, less dependent on a good mechanical contact with the patient's skin, and an ultrasonic Doppler detector which is more cumbersome but provides a stronger, more robust signal.

In any case, if the Doppler detector is attached to the needle tip protector 28 (FIG. 2A), both the tip protector and Doppler detector should be removed prior to inserting the needle into the skin. Conveniently the needle tip protector may incorporate a slit so that, once the search for a vein is complete, the needle tip protector can be removed from the tip of the needle and attached to the rear part of the fiber distal fitting 25 (FIG. 2A). In this manner the needle tip protector and the Doppler detector, which are connected to the device with the cable 43, do not interfere with the continued needle insertion procedure.

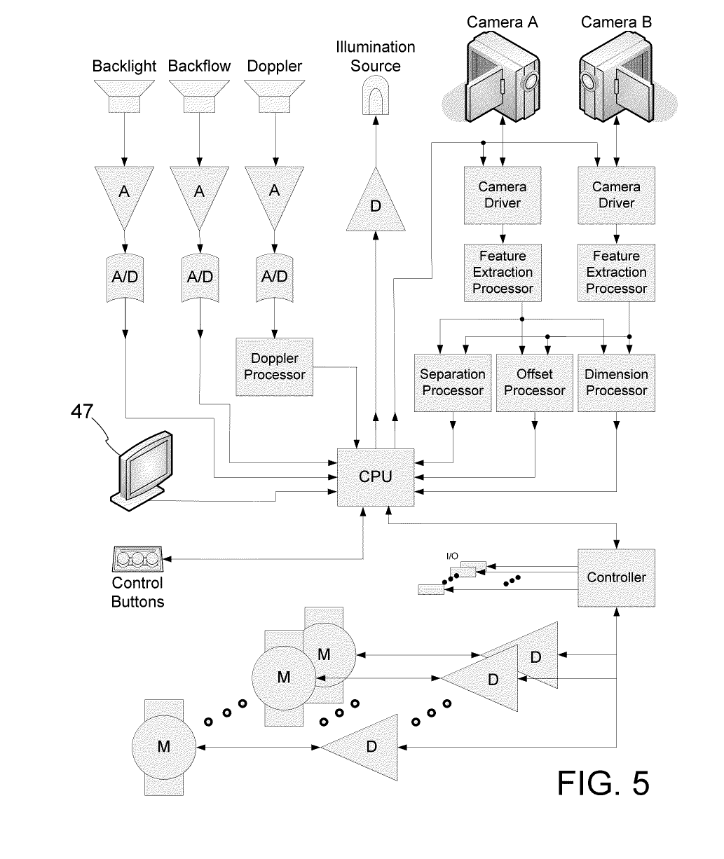

An alternative system 45 is shown schematically in FIG. 4C. Here the needle/catheter/optical fiber assembly 10 is also deployed manually but the veins are viewed with the aid of a camera 46 that images the veins as illuminated with the light emanating from the tip of the needle. The image picked up by the camera is conveniently displayed for the operator on a suitable screen 47 (shown in FIG. 5), or can be conveniently projected on to a surface nearby, or even directly on to the patient's skin. Optionally such a screen can be that of a personal “smart” mobile phone or tablet type computer, and the communication between camera and such display based on a short-range wireless channel (if allowed in the relevant operating environment). Using a camera permits illumination with red or NIR light, or a combination of the two. The illumination source and the camera may be battery operated. Optionally the source is polarized and the camera supplied with an orthogonal polarizer. In this case, the system may optionally include a polarization controller so that the operator can optimize the contrast of the vein images (not shown). FIG. 4C shows the system in the first phase of the operation—searching for a suitable vein. The additional steps of the operation, shown in FIG. 2B through 2D are performed in a manner similar to the description related to these figures. The optional Doppler detector 42, may be located on the needle/catheter/optical fiber assembly as described above with reference to FIG. 4B, or alternatively be co-located with the camera 46. The result of the Doppler measurement may be conveniently displayed on the same screen displaying the camera image. Optionally the Doppler detector may be detachable from the camera 46 to allow it to be used in contact with the skin.

The camera 46 described above may be conveniently worn by the operator, for example with suitable head-gear, leaving the operator's hands free to manipulate the needle/catheter/optical fiber assembly. Alternatively the camera can be mounted on a suitable fixture, attached to the surface on which the patient is positioned, (arm-chair, stretcher or bed) to conveniently display the image of the veins. Alternatively the camera can be mounted on a medical cart to offer convenient mobilization on the one hand and convenient positioning over the relevant area of skin on the other hand. A further alternative is considered below (FIG. 4G) where the camera is attached to the needle/catheter/optical fiber assembly 12.

Still an alternative system 50 is described schematically in FIG. 4D. Here the needle/catheter/optical fiber assembly 12 is also deployed manually but the veins are viewed with the aid of two cameras 46a and 46b proving a perspective 3D image of the veins as illuminated with the light emanating from the tip of the needle. Preferably the two cameras are mounted in a fixed assembly to ensure that the perspective view is maintained constant. The image picked up by the cameras is conveniently displayed for the operator on a suitable screen, or can be conveniently projected on to a surface nearby or even directly on to the patient's skin. As noted above, such a screen can be a mobile phone or a tablet computer. As before, using cameras permits illumination with red or NIR light, or a combination of the two. The illumination source and the camera may be battery operated. Optionally the source is polarized and the cameras supplied with orthogonal polarizers 22. It will be understood that the polarizers 22 are shown schematically since in practice they must obviously cover the lens or be incorporated within the camera. In this case, the system may optionally include a polarization controller (not shown) placed on the illuminating fiber 15 or on the polarizers of the cameras so that the operator can optimize the contrast of the vein images. FIG. 4D shows the system in the first phase of the operation—searching for a suitable vein. The additional steps of the operation, shown in FIG. 2B through 2D are performed in a manner similar to that described with reference to these figures. As above, the optional Doppler detector 42 may be located on the needle/catheter/optical fiber assembly, or co-located with one of the cameras 46a or 46b, and the results displayed on the same screen displaying the camera image. Optionally the Doppler detector 42 may be detachable from the camera to allow it to be used in contact with the skin. As above, the camera assembly may be wearable, or mounted on to the working surface or mounted in a medical cart or on to the needle/catheter/optical fiber assembly 12.

A fifth alternative system 55 is described schematically in FIG. 4E, which includes, in addition to the system of FIG. 4D, an automated, multi-axis, mechanical needle guide system having an automatic 5-axis manipulator 57. The needle/catheter/optical fiber assembly 12 is inserted manually, with the aid of a needle insertion slide 58 mounted on the 5-axis manipulator 57, which controls the lateral position of the needle along two linear axes, x and y, and its azimuth and elevation with two rotational axes, θ and φ, respectively. The manipulator guide is controlled by a suitable processor 59 that in turn receives geometrical aiming information from automatic image processing of the 3D image obtained with the two cameras 46a and 46b. The operator is required to verify the automatic aim of the system and manually guide the needle/catheter/optical fiber assembly along an insertion axis that is collinear with the needle insertion slide 58. In addition the operator can manually control the rotational axis, Ω which rotates the hypodermic needle device 10 about an axis along the length of the needle. The latter motion is important for addressing problematic rolling veins, and to reduce the likelihood of damage to the back wall of the vein. The problems of rolling veins flexing away from the contact with the tip of the needle are addressed by rotating the needle about its axis to improve the piercing ability of the needle tip. In contrast, to prevent accidental piercing of the back wall of the vein, the needle can be rotated so that the tip of the needle is in a less favorable orientation for penetration. Alternatively the motion in the Ω axis can be preset, either with a mechanical spring (described above with reference to FIGS. 3J through 3L) or with electronic control, to rotate by 90° or 180° in one step on indication of vein wall penetration. Such large-step rotation can be activated manually or with an automated command

An additional optional feature relates to automatic release of the needle and fiber assembly after insertion into the vein. Here a second spring-loaded mechanism 36 is provided as shown in FIG. 4F. This mechanism can be operated manually by releasing a latch, or automatically, activated by the backflow signal from the backflow detector 33 (with a suitable delay to separate it from any automated motion of the spring loaded Ω-axis rotation 34.

A sixth alternative system 60 is shown schematically in FIG. 4G, in which miniature cameras 61a and 61b for viewing the veins are mounted directly on to the needle/catheter/optical fiber assembly 12. A basic system includes a single camera 61a which operates similarly to the external camera 46 described above in connection with FIG. 4C to visualize veins. Such a camera permits the use of NIR illumination which cannot be viewed with the unaided eye. The miniature two-camera option as shown in FIG. 4G offers, in addition, 3D imaging of the veins, similar to the description corresponding to FIG. 4D above. Suitable miniature cameras are currently available for application in mobile phones and endoscopes. Each camera is mounted on a respective camera fixture 62, which is detachable from the needle/catheter/optical fiber assembly 12. Each camera with its respective fixture is therefore part of the supporting equipment that is not disposable and not replaced between procedures (as is the illumination source). In other words, the camera-fixture is mounted on to a disposable needle/catheter/optical fiber assembly before every procedure. The camera fixture is removed from this assembly once the catheter is in place in the vein, together with the needle and optical fiber. It is separated from the needle and optical fiber when these are discarded and prepared for use in the next procedure.

As in the descriptions above, in the system 60, the needle/catheter/optical fiber assembly 12 is also deployed manually, the veins being viewed with a screen. Polarization control may optionally be included and the system can optionally be battery powered all similar to the descriptions above.

Yet another alternative system (not shown) is intended for completely automated insertion of an IV catheter. The automated system comprises a seven-axis robotic manipulator adapted to hold and position a needle/catheter/optical fiber assembly 12 in space; a fixture to position and stabilize the patient's relevant organ, for example a forearm, for the duration of the procedure; a Doppler detector (either optical or ultrasonic); an optional blood backflow detector; an illumination source and a central processor to process the visualized vein images. Six of the seven robotic axes are: three linear, similar to the axes x, and y in FIG. 4E, plus a vertical axis z normal to the skin; azimuth and elevation axes, similar to the axes θ, φ in FIG. 4E; and an axial rotation axis about the length of the needle, similar to axis Ω in FIG. 4E. In addition the manipulator includes a seventh, linear axis along the length of the needle, called the I-axis. This axis controls the insertion of the needle/catheter/optical fiber assembly 12 along an automated axis, similar to the manual guide shown in FIG. 4E. In addition automated fixtures are provided to fix the catheter in place once inside the vein and remove the needle and optical fiber. The cameras, the fiber needle tip illuminator and the Doppler detector are operable similar to their operation in the manual procedures describe with relation to FIGS. 4B, 4D and 4G with the various options and alternatives described there.

The processor is adapted to receive perspective 3D images from the cameras and analyze them to obtain the different information required for the different phases of the procedure described in relation to FIGS. 2A through 2D and move the needle/catheter/optical fiber assembly accordingly through control of the seven robotic axes provided. Specifically, once the patient's forearm, for example, is secured in its fixture, the processor can control the system to scan possible veins searching for a suitable vein. For this purpose the vein camera images provide information on the size, orientation and topology of different veins to seek a vein with a relatively large size, and a straight extent with no bifurcations. Using the data from the Doppler detector the processor can determine that the selected vein is patent (with active blood flow). In this manner a suitable vein for inserting the catheter can be located automatically. In the second phase of the procedure, the robotic manipulator positions the needle/catheter/optical fiber assembly at the appropriate orientation for penetrating the selected vein. The processor continuously monitors the relative locations of the vein and the tip of the needle, confirming the needle trajectory is in the correct direction both in its lateral displacement as well as in its relative distance. The processor can rotate the needle in the Ω axis to counter vein rolling. On first penetration into a vein either an optical signal (in the form of significant change in back-reflected light, and a significant reduction in the illumination halo emanating from the fiber tip), and\or an indication for backflow (with an additional sensor introduced to identify the presence of blood in the proximal cavities of the needle), at which point the insertion process is halted, the catheter fixed in place and the needle/optical fiber components of the assembly removed.

Naturally such a fully automate system offers a continuous display of the process on a suitable screen with options for manual over-ride.

FIG. 5 is a schematic block diagram of a fully automated version of the system described above. Corresponding block diagrams of the previous systems, with lesser degrees of automation, can be inferred from that of the complete system. The system includes five sensing devices, shown at the top of FIG. 5. These include, from left to right, a light sensor for back-reflected light in the needle; a light sensor for the presence of blood in the backflow chamber in the proximal end of the needle; a Doppler detector, and two cameras. The signal from the light sensors, which are photodetectors sensitive to the wavelength of the illumination source, such as silicon photedetectors, is amplified by amplifier A, filtered (not shown), and converted to digital form in an A/D converter. Optionally the backflow detector includes an illumination source to improve the optical signal (not shown). The signals in digital form are fed into the central processing units (CPU). The signal generated by the Doppler detector, which may be a light sensor or an ultrasonic sensor, is amplified with amplifier A, filtered (not shown), digitized with an A/D converter, and fed into a Doppler processor which extracts the Doppler signal from the sensor signal. In the case of an ultrasonic Doppler sensor, the sensor is driven to transmit ultrasound that serves as the carrier signal to be modulated by the blood flow (not shown). This signal is pulsating according to the heart beat of the patient. From this signal it is possible to extract a measure of the flow rate in the vein, as well as the heart-beat rate. These measurements are fed as inputs to the CPU. Each of the cameras is controlled by a camera driver which serves to automatically setup various parameters of the camera (such as exposure time, integration rate etc.), and extract the images obtained in real time. The images of each camera are fed into a feature extraction processor where the outline of veins and needle tip are accentuated. These processors, as well as the processors described below, can be separate dedicated hardware processors, or, alternatively may be integrated within a larger processor, or implemented as sub-processes or software routines within one or more hardware processors. The extracted features are fed in parallel to three dedicated processors designed to calculate from the perspective of the two camera images: a separation processor determines the distance between the needle tip and vein; an offset processor determines the lateral offset and its direction between the needle tip and the vein; and a dimension processor determines the dimensions of the vein itself. The results of all the above processors are fed into the CPU.

In addition to the control of the camera driver, the CPU commands the illumination source on and off via a driver D. One exemplary source is shown in FIG. 5, representing a possibility of several such sources. The illumination source, or sources, may include more than one wavelength and/or illumination in more than one fiber. The CPU controls the timing of switching on the illumination of each of the sources to improve the visibility of the veins in the image. The CPU can also control the polarization of each source via the motor controller described below.

There are several motorized axes in the system. As described above there are five positioning axes, x, y, z, θ and φ. In addition there is the needle rotation axis Ω and the needle insertion axis I. To these are added the fiber polarization control motors. All these motors, M in the figure, are driven by motor drivers D, and controlled by the axes-controller (Controller). In addition the Controller can operate valves and pistons with its electrical input/output ports (I/O). These serve to activate pistons and latches and similar devices, such as needed to remove the needle and fiber after penetrating a vein.

Finally the CPU drives a display 47 where the status of the operation is presented, including display of images of veins and the location of the tip of the needle. Optionally a 3D screen can be used. The user is offered adjustments and over-ride of different functions with the aid of several control buttons (Control Buttons) including the possibility of touch-screen functions and use of a pointing device for selecting and marking functions and data on screen.

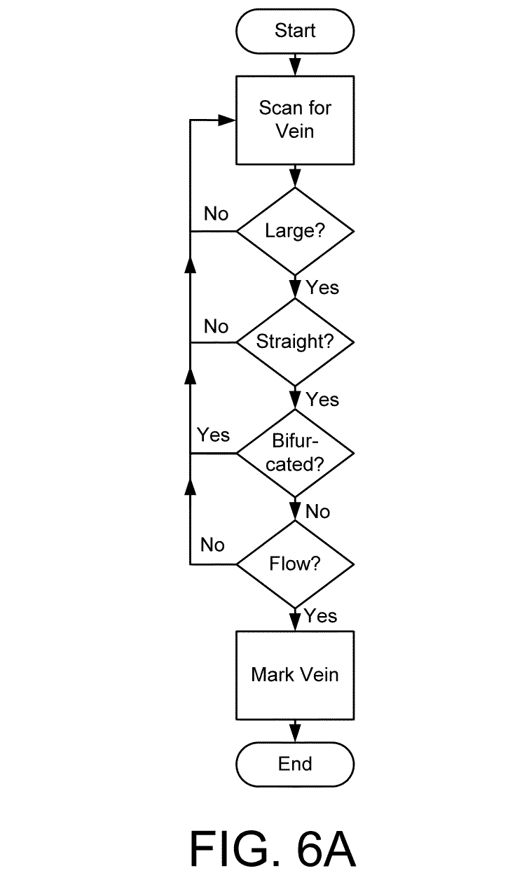

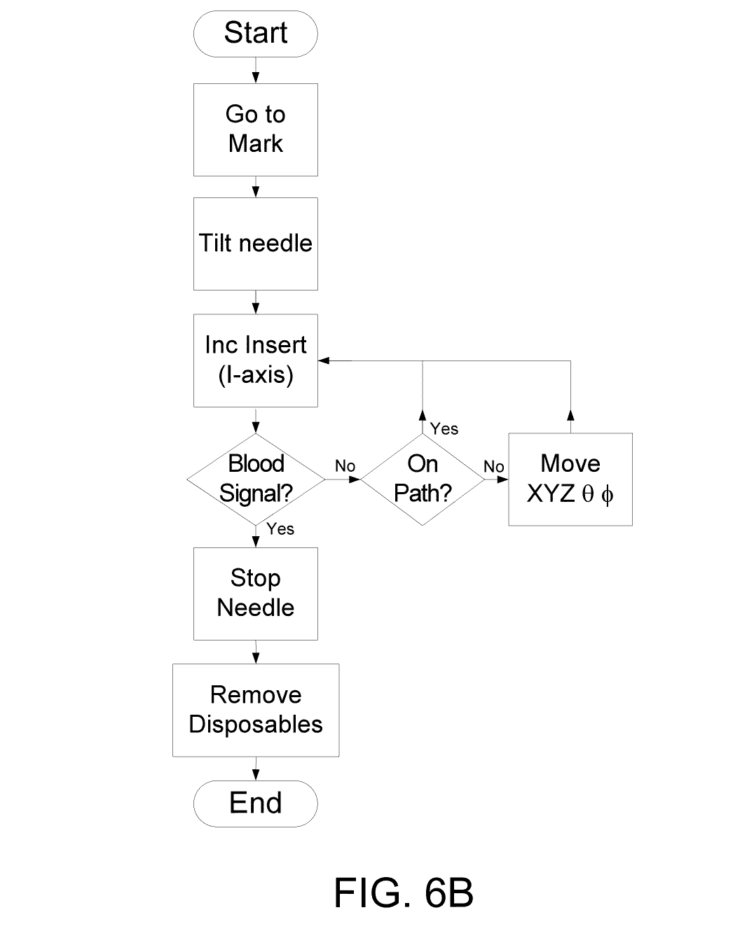

FIGS. 6A and 6B show schematically the flow chart of the two phases of operations described above, the search phase, and the insertion phase, respectively. These apply equally well to manual as well as fully automated procedures. The following describes the automated procedure in correlation with the various functional blocks of the system as described above with reference to FIG. 5. In the search phase, the image captured by the cameras is transferred through the camera drivers to the feature extraction processor and to the dimension processor. In the search phase it is the dimension processor which generates the data on the size of the vein in the camera image, and its topology (if it is straight and, if so, whether it is also bifurcated). This data is fed to the central processing unit (CPU) to allow it to perform its search process. The inputs from the Doppler sensor and back-reflection and backflow detectors are also input to the CPU.

The search process (FIG. 6A) is initiated by command from the control buttons once the patient is secured in a stabilizing fixture (Start). The CPU controls the axes to scan the patient's skin. When a vein is identified in the feature extraction processor, scanning is halted and the dimension processor transfers the data to the CPU to determine whether the vein is large, straight and not bifurcated. In this case the CPU also checks the Doppler detector signal. If that signal shows a good blood flow rate, that position is marked for piercing the vein. This entire procedure may be repeated several times (not shown in FIG. 6A) to scan for different veins. Once several acceptable veins are found, the one that registers the best parameters is selected, its position is marked and the search phase of the process ends.

The piercing phase of the process (FIG. 6B) is initiated by command from the control buttons while the patient is still secured in a stabilizing fixture (Start). The CPU controls the axes to move the needle to the selected vein location (Go to Mark) then tilt the needle for a near-flat entry angle (Tilt Needle) and increment the needle insertion axis [Inc Insert (I-axis)]. After each I-axis increment, the CPU monitors the back-reflection and the backflow light sensors to ensure that the vein has not been penetrated yet (Blood Sig?), then addresses the lateral offset and the needle tip to vein distance to determine if the I-axis increment has been on path (On path?). If required to return to the path, (On path, “No” branch) the manipulation axes x, y, z, θ and φ are controlled via the axes controller to bring the needle closer to its penetration path. Consequently the next I-axis increment is performed. This process is repeated until indication is received for penetration of the vein, either with the back-reflection or backflow sensors. At this point the I-Axis motion is halted (Stop Needle) and the needle may optionally be rotated about its Ω-axis (not shown in the flow chart). Finally the needle and fiber assemblies are removed through a command to the I/O devices activating the piston required to remove the disposable components, and the process ends.

The description of the above embodiments is not intended to be limiting, the scope of protection being provided only by the appended claims.

In particular it should be noted that features that are described with reference to one or more embodiments are described by way of example rather than by way of limitation to those embodiments. Thus, unless stated otherwise or unless particular combinations are clearly inadmissible, optional features that are described with reference to only some embodiments are assumed to be likewise applicable to all other embodiments also.

1.-45. (canceled)

46. A hypodermic needle device, comprising:
a hypodermic needle having a tubular bore;
at least one optical waveguide extending along through said needle or along an exterior wall thereof so that a distal end of the at least one optical waveguide is in proximity to a tip of the needle;
a first coupler for securing the at least one optical waveguide within the needle at the proximal end of the needle; and
a second coupler for removably securing a proximal end of the at least one optical waveguide to a respective illumination source in order that light will emanate from the distal end of the at least one optical waveguide;
wherein the second coupler contains a lens for focusing light from the illumination source through the at least one optical waveguide; and
wherein the second coupler is remote from the first coupler.

47. The device as claimed in claim 46, wherein said at least one optical waveguide is the tubular bore of said needle.

48. The device as claimed in claim 46, wherein said at least one optical waveguide comprises at least one optical fiber extending such that the distal end of each optical fiber is located closer to the proximal end of the needle and from there the internal surface of the needle serves as the waveguide so that illumination exiting the distal end of each fiber is further guided by internal surfaces of the tubular bore to emanate from the tip of the needle.

49. The device as claimed in claim 48, wherein said first coupler is configured to allow removal of the at least one optical fiber from the tubular bore.

50. The device as claimed in claim 46, wherein the at least one optical waveguide is at least one single strand optical fiber located outside the tubular bore of said needle and attached to an outer surface of the needle with the aid of an outer sleeve located over the needle and optical fiber for securing the optical fiber.

51. The device as claimed in claim 50, wherein the at least one optical fiber is located in a respective longitudinal groove formed in the outer surface of the needle.

52. The device as claimed in claim 46, wherein said at least one optical waveguide comprises at least one optical fiber embedded into the wall of a tubing sleeve surrounding and attached to the outer surface of the needle extending such that the distal end of each optical fiber is located at a distance from the distal end of the tubing sleeve, the tubing sleeve being disposed substantially proximate the distal end of the needle such that the internal surface of the tubing walls serve as the waveguide so that illumination exiting the distal end of each fiber is further guided by internal surfaces of the tubing sleeve to emanate from the tip of the needle.

53. The device as claimed in claim 51, wherein more than one said optical fiber is attached to the outer surface of the needle, and wherein each optical fiber is secured by the first and second couplers.

54. The device as claimed in claim 46, wherein the at least one optical waveguide is a disposable optical fiber that is removable from its respective illumination source by means of the second coupler and from the needle by means of the first coupler.

55. The device claim 46, wherein said illumination sources are any of the following: a solid state laser, a fiber laser, a semiconductor laser or a LED.

56. The device as claimed in claim 46, wherein at least one of said illumination sources emits red or near infrared (NIR) light.

57. The device as claimed in claim 46, further including a beam splitter inside the second coupler for redirecting backscattered light on to a photodetector that is adapted to monitor intensity of the backscattered light so as to identify a reduction in said intensity consequent to penetration of the needle into a vein.

58. The device as claimed in claim 46 wherein the illumination source directs red or NIR light through the at least one optical waveguide such that red or NIR light illumination emanates from a tip of the needle, and further including a manually adjustable polarizer for optimal vein image contrast.

59. The device as claimed in claim 57, further including a backflow detector to monitor a presence of blood in the backflow chamber of the needle.

60. The device as claimed in claim 46 including at least two light sources configured to illuminate at different wavelengths coupled into at least one optical fiber.

61. The device as claimed in claim 46, wherein a tip of the needle is chamfered and there is further included a spring-loaded mechanism for rotating the needle 180° about a longitudinal axis (Ω) of the needle.

62. A system comprising:
an assembly comprising a hypodermic needle having a tubular bore and a catheter;
at least one optical waveguide extending along said needle so that a distal end of the at least one optical waveguide is in proximity to a tip of the needle;
a first coupler for securing the at least one optical waveguide within the needle at the proximal end of the needle, at least one illumination source optically coupled to inject light directly into a proximal end of the needle, such that light emanates from a distal end of the optical waveguide;
a second coupler remote from the first coupler for removably securing the at least one optical illumination source to the needle; and
a lens within the second coupler for focusing light from the at least one illumination source through the at least one optical waveguide.

63. The system as claimed in claim 62, wherein said at least one optical waveguide is an optical fiber that runs through the tubular bore of said needle or is mounted on an external surface thereof.

64. The system as claimed in claim 62, wherein at least one of said illumination sources emits red or near infrared light.

65. The system as claimed in claim 62, further including a photodetector for receiving backscattered light redirected by a beam splitter inside the second coupler and being adapted to monitor intensity of the backscattered light so as to identify a reduction in said intensity consequent to penetration of the needle into a vein.

66. The system as claimed in claim 62, including a photodetector and electronic circuitry for monitoring backscattered light from a vein, said electronic circuitry being adapted to measure a Doppler shift in the backscattered light and output an indication of the corresponding flow rate in a monitored vein.

67. A system for NIR vein visualization comprising:
a hypodermic needle device including:
a hypodermic needle having a tubular bore;
at least one optical waveguide extending along through said needle or along an exterior wall thereof so that a distal end of the at least one optical waveguide is in proximity to a tip of the needle;
a first coupler for securing the at least one optical waveguide within the needle at the proximal end of the needle; and
a second coupler for removably securing a proximal end of the at least one optical waveguide to a respective illumination source in order that light will emanate from the distal end of the at least one optical waveguide;
wherein the second coupler contains a lens for focusing light from the illumination source through the at least one optical waveguide; and
wherein the second coupler is remote from the first coupler;
at least one NIR camera disposed to image light scattered from tissue illuminated by the needle before and after said needle is injected into said tissue;
a polarizer disposed in front of the camera and being manually adjustable for optimal vein image contrast; and
a display coupled to the at least one NIR camera for presenting an image of said vein.
